(19) United States
(12) Patent Application Publication
(10) Pub. No.: US 2022/0196832 A1
(43) Pub. Date: Jun. 23, 2022

(54) SYSTEMS AND METHODS FOR CONTACTLESS MOTION TRACKING

(71) Applicant: University of Washington, Seattle, WA (US)

(72) Inventors: Shyamnath Gollakota, Seattle, WA (US); Anran Wang, Seattle, WA (US); Jacob Sunshine, Seattle, WA (US)

(73) Assignee: University of Washington, Seattle, WA (US)

(21) Appl. No.: 17/604,311

(22) PCT Filed: Apr. 16, 2020
(86) PCT No.: PCT/US2020/028596

§ 371 Date: Oct. 15, 2021

Related U.S. Application Data
(60) Provisional application No. 62/834,706, filed on Apr. 16, 2019.
(60) Provisional application No. 62/911,502, filed on Oct. 7, 2019.
(60) Provisional application No. 62/911,872, filed on Oct. 7, 2019.

(51) Int. Cl. G01S 15/58; G01S 15/66; G01S 15/88; A61B 5/11; A61B 5/113; A61B 7/00; A61B 5/0205; A61B 5/08; A61B 5/00

(52) U.S. Cl. CPC G01S 15/586; G01S 15/66; G01S 15/88; A61B 5/1113; A61B 5/4806; A61B 7/003; A61B 5/0205; A61B 5/0816; A61B 5/113

(57) ABSTRACT

Embodiments of the present disclosure provide systems and methods directed to contactless motion tracking. In operation, a speaker may provide an acoustic signal to, for example, a subject. A microphone array may receive a reflected acoustic signal, where the received reflected signal is responsive to the acoustic signal reflecting off the subject. A computing device may extraction motion data of the subject based on the received reflected acoustic signal. Various motion data extraction methods are described herein. The motion data may include respiration motion, coarse movement motion, respiration rate, and the like. Using the extracted motion data, the processor may identify at least one health condition and/or sleep anomaly corresponding to the subject. In some examples, beamforming is implemented to aid in contactless motion tracking.

### CROSS-REFERENCE TO RELATED APPLICATION

This application claims the benefit under 35 U.S.C. § 119 of the earlier filing dates of U.S. Provisional Application Ser. No. 62/834,706 filed Apr. 16, 2019, U.S. Provisional Application Ser. No. 62/911,502 filed Oct. 7, 2019, and U.S. Provisional Application Ser. No. 62/911,872 filed Oct. 7, 2019, the entire contents of each are hereby incorporated by reference in their entirety for any purpose.

### STATEMENT OF FEDERALLY SPONSORED RESEARCH AND DEVELOPMENT

This invention was made with government support under Grant. No. 1812559, awarded by the National Science Foundation. The government has certain rights in the invention.

### TECHNICAL FIELD

Examples described herein generally relate to contactless motion tracking. Examples of extracting motion data of a subject using signal reflections and in some cases using receive beamforming techniques are described.

### BACKGROUND

Sleep plays an important role in obtaining and maintaining good health and overall well-being. For example, sleep aids in learning and memory by helping the brain commit new information to memory, contributes to metabolism and weight management by affecting the way the body processes food and alters hormone levels, and is linked to cardiovascular health and immune function. Sleep is also vitally important for neurological development in children, and particularly infants. While getting enough sleep is important to overall well-being, how an individual sleeps is equally as important, and may be indicative of underlying, often devastating, health conditions.

Consumer sleep products that monitor vital signs, movement, noise, and the like during sleep have become increasingly popular. For example, many adults use sleep monitoring devices (e.g., rings, watches, straps, bands, mats, etc.) to track various sleep data such as heart rate, sleep time, and snore duration, to get a better gauge of their overall health. Athletes too have turned to sleep monitoring for tracking various sleep data such as heart rate variability (HRV) to help determine over-training, athletic condition, athletic performance, and sleep-based recovery. For children, however, many caregivers turn to specialized infant monitors (e.g., invasive vital sign tracking systems) that clinically track essential body function such as respiratory rates, especially for children less than one year of age, because of their susceptibility to rare and devastating sleep anomalies, such as, for example, Sudden Infant Death Syndrome (SIDS).

The use of modern technologies and medical advancement in sleep tracking by way of consumer sleep products has made possible the monitoring of vital signs, movement, noise, and the like while sleeping, which may be indicative of underlying health conditions. However, while these technologies may help with some level of sleep tracking, there still exists challenges in effetely tracking a more comprehensive set of sleep data (e.g., minute breathing, respiration rate, limb and/or other movement, noise, etc.), while doing so in an noninvasive (e.g., no wires, no wearables, etc.) manner.

### SUMMARY

Embodiments described herein are directed towards systems and methods for contactless motion tracking. In operation, a speaker may provide a pseudorandom signal. In some embodiments, the pseudorandom signal may comprise an acoustic signal. In some embodiments, the pseudorandom signal may comprise at least one of a white noise signal, a Gaussian white noise signal, a brown noise signal, a pink noise signal, a wi de-band signal, a narrow-band signal, or combinations thereof. In some embodiments, the pseudorandom signal may comprise at least one of an audible signal, an inaudible signal, or a combination thereof. In some embodiments, the speaker may generate the pseudorandom signal, based, at least in part, on a phase-shift encoded impulse signal.

A microphone array may receive a reflected pseudorandom signal based at least on the provided pseudorandom signal, where the received reflected pseudorandom signal is responsive to the provided pseudorandom signal reflecting off a subject. In some embodiments the subject may be a motion source or an environmental source.

A processor may extract motion data of the subject, based at least in part, on the received reflected pseudorandom signal. In some examples, the motion data may comprise at least one of a respiratory motion signal, a coarse movement motion signal, a respiration rate, a health condition, or a combination thereof.

In some embodiments, the processor may extract the motion data based further on transforming the received reflected pseudorandom signal into a structured signal (e.g., an FMCW signal, FMCW chirp), where the transforming is based, at least in part, on shifting a phase of each frequency component of the received reflected pseudorandom signal; demodulating the structured signal, where the demodulating is based, at least in part, on multiplying the structured signal (e.g., structured chirp) by a conjugate signal (e.g., a downchirp in case the pseudorandom signal is transformed to a structured signal that is an upchirp), where the demodulating results in a demodulated signal (e.g., demodulated chirp) and at least one corresponding frequency bin; decoding the demodulated signal (e.g., demodulated chirp), where the decoding is based, at least in part, on performing a fast Fourier transformation (FFT) on the demodulated signal (e.g., demodulated chirp), resulting in at least one corresponding FFT frequency bin; and extracting, using phase information associated with the corresponding FFT frequency bin, the motion data of the subject.

In some embodiments, the processor may extract the motion data based at least on determining a value of a FFT frequency bin corresponding to an estimated round-trip distance of the received reflected pseudorandom signal; using the value of the FFT frequency bin, determine a respiratory motion signal; and applying sub-band merging and phase shift compensation to extract a continuous phase signal.

In some embodiments, the processor may extract the motion data based at least on feeding amplitude information corresponding to the received reflected pseudorandom signal into a neural network, where the neural network is configured to compress the amplitude information from a two-dimension (2D) space into a one-dimensional (1D) space; and based at least on the compressed amplitude information, extracting the motion data of the subject. In some examples, the neural network may comprise at least one of a convolutional neural network, a deep convolutional neural network, a recurrent neural network, or combinations thereof.

In some embodiments, the processor may synchronize the speaker and the microphone array, based at least in part on regenerating the provided pseudorandom signal using a known seed, performing cross-correlation between the received reflected pseudorandom signal and the regenerated provided pseudorandom signal, where the performing results in a cross-correlation output, and identifying a peak of the cross-correlation output, where the peak corresponds to a direct path from the speaker to the microphone array.

In some embodiments, the processor may localize the subject based at least in part on determining a distance from the speaker to the subject. In some embodiments, the processor may localize the subject further based on beamforming the received reflected pseudorandom signal to generate a beamformed signal, and determining a location of the subject, based at least in part, on the beamforming.

In some examples, the processor may identify at least one e th condition based at least on extracting the motion data of the subject.

Additionally, embodiments described herein are directed towards systems and methods for contact less motion tracking using receive beamforming. In operation, a speaker may provide an acoustic signal. A processor may perform receive beamforming based at least on a determined distance between a subject and the speaker, a determined beamforming signal, a determined angle of the subject relative to the speaker, or a combination thereof. A microphone array may receive a reflected acoustic signal based on the acoustic signal reflecting off the subject. The processor may extract motion data of the subject based at least in part, on the received reflected acoustic signal.

In some embodiments, determining the angle of the subject relative to the speaker is based at least on performing a search over multiple angles to locate a selected angle based on a signal strength of the motion data. In some embodiments, determining the angle of the subject relative to the speaker is based at least on a ternary-search performed by changing a search range as well as a beam width to compute a direction of the subject. In some embodiments, determining the angle of the subject relative to the speaker is based at least on a computation that starts at lower frequencies to reduce an effect of direction for the subject, and utilizes higher frequencies to increase beam resolution and select a direction of the subject.

### BRIEF DESCRIPTION OF THE DRAWINGS

Reference is now made to the following descriptions taken in conjunction with the accompanying drawings, in which:

FIG. 1 is a schematic illustration of a system for contactless motion tracking, arranged in accordance with examples described herein;

FIG. 2 illustrates a schematic illustration of using contactless motion tracking results for identification of health conditions and medical correction, in accordance with examples described herein;

FIG. 3 illustrates a flowchart of a method for contactless motion tracking, in accordance with examples described herein; and

FIG. 4 is a flowchart of a method for contactless motion tracking using beamforming techniques, arranged in accordance with examples described herein.

### DETAILED DESCRIPTION

The following description of certain embodiments is merely exemplary in nature and is in no way intended to limit the scope of the disclosure or its applications or uses. In the following detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

Various embodiments described herein are directed to systems and methods for improved contactless motion tracking. Contactless motion tracking may include, but is not limited to tracking respiratory motion, coarse movement motion (e.g., arm movement, leg movement, head movement, etc.), respiration rate, and the like, of a subject. In some examples, receive beamforming techniques may be implemented to aid in contactless motion tracking. The phrase contactless motion tracking is used to indicate that motion-related data may be obtained using systems and techniques described herein without physically contacting a subject with a probe or other adhered or attached sensor. It is to be understood, however, that the contactless motion tracking systems and techniques described herein may in some examples include one or more contact sensors that may augment, accompany, and/or enhance contactless measurements. In some examples a speaker, such as a white noise machine, may provide an acoustic signal. In some examples, the acoustic signal may be a pseudorandom signal, such as a Gaussian white noise signal. A microphone array may receive an acoustic signal reflected from a subject (e.g., a motion source, an environmental source, etc.), based at least in part on the provided acoustic signal. In some examples, receive beamforming techniques may be used to aid in the detection of the reflected acoustic signal. A processor may be used to extract motion data from the subject based in part on the received reflected acoustic signal. Various example techniques for extracting motion data from the subject are described herein. Using the extracted motion data, various health conditions may be identified, such as cardiac-related health conditions, congenital ENT anomalies, gastrointestinal-related health conditions, as well as neurological- and musculoskeletal-related conditions, etc.

Currently available motion tracking systems may suffer from a number of drawbacks. With respect to adults, motion data is often tracked using smartwatches, Bluetooth-enabled bracelets, rings, as well as bedside and bed-integrated devices. While such devices may be may enable general sleep hygiene and sleep habit tracking, they often lack reliability, accuracy, and are limited in what motion they can track. For example, many current sleep trackers for adults use motion-sensing technology such as accelerometers and/or gyrometers to gauge how often a wearer moves during sleep. Data gleaned from such sensors is often inaccurate, and may over and/or underestimate motion data. Moreover, and particularly with respect to wearable devices, such devices may be obtrusive to the wearer, and may prevent the wearer from falling and/or staying asleep. Further, even if these devices were able to accurately track motion in adults, they lack universality and are age-restrictive. In other words, such motion tracking systems lack accuracy and reliability if attempted to use for infants and young children.

With respect to children, and particularly infants, current motion tracking systems (e.g., vital sign tracking monitors) are almost exclusively contact-based systems, which are often prohibitively invasive. For example, some devices currently used to track infant vital signs during sleep use specifically designed sensors and wires that often require contact with the infant or with the infant's sleep surface. Not only do these contact-based systems often prevent, as well as cause discomfort during, sleep, they have also lead to severe complications, such as, for example, rashes, burns, and death from strangulation. Additionally, current motion tracking systems for infants are often limited by what they can and cannot monitor, as well as suffer from a lack of reliability and accuracy in their results.

Even further, with respect to children, and particularly infants, speakers (e.g., white noise machines, other machines capable of providing acoustic signals and/or pseudorandom signals, etc.) are often used to achieve faster fall asleep times, attain longer sleep times, and improve overall sleep quality for infants. However, while such white noise speakers are used to improve quality of sleep, they are currently unable to monitor and/or track motion. For example, pseudorandom signals (e.g., white noise) are random in both the time and frequency domain. As a result, it is often challenging to embed or extract useful information from white noise signals. Moreover, the signal strength of the reflected signal off of a subject (e.g., infant) that correspond to, for example, respiratory motion (e.g., breathing) is generally proportional to the surface area of a subject's chest. Because infants have considerably smaller torsos compared to adults as well as their chest displacement due to breathing is also much smaller compared to adults, it is often challenging to detect and extract information (e.g., motion data) from such reflected signals.

Additionally, many current motion tracking systems that work for both adults and children may be cost-prohibitive and may require physician-operated equipment. For example, a polysomnogram (e.g., a sleep study) is an overnight comprehensive sleep test that monitors brain waves, breathing, heath rhythm, oxygen levels, and muscle tone. Such a test may be used to track motion and in some cases identify sleep disorders. However, while such tests are compressive, they often require specifically designed sensors and wires that involve contact with the sleep study participant that are both obstructive and invasive, they require the participant to stay overnight at a medical facility for the duration of the test to be continuously monitored, and such a study cannot be used to track motion on a daily basis from within a participant's own bed. Further, often times to participate in a polysomnogram sleep study, a prescription or physician referral is required, and such tests are often prohibitively expensive.

Accordingly, embodiments described herein are generally directed towards contactless motion tracking. In this regard, embodiments described herein enable contactless motion tracking by providing an acoustic signal, and a receiving a reflected acoustic signal based on the provided acoustic signal reflecting off a subject, such as, for example, a motion source (e.g., a person), an environmental source (e.g., furniture, a plant, walls, etc.), or a combination thereof. In some examples, receive beamforming techniques may be used to aid in the detection of the reflected acoustic signal. Motion data (e.g., respiratory motion, coarse movement motion, respiration rate, and the like) may be extracted from the subject using the received reflected acoustic signal based at least on various extraction techniques described herein. Using the extracted motion data, various health conditions may be identified, such as cardiac-related health conditions, congenital ENT anomalies, gastrointestinal-related health conditions, as well as neurological- and musculoskeletal-related conditions, etc.

In some embodiments, a speaker (e.g., a white noise machine, a smart speaker, etc.) may provide an acoustic signal. In some embodiments, the acoustic signal may be a pseudorandom signal, such as, for example, a white noise signal, a Gaussian white noise signal, a brown noise signal, a pink noise signal, a wide-band signal, a narrow-band signal, or any other pseudorandom signal. In other examples, the acoustic signal may be audible, inaudible, or a combination thereof.

Examples of a microphone array described herein may receive a reflected acoustic signal, where the reflected acoustic signal received is responsive to the provided acoustic signal reflecting off a subject, such as, for example, a motion source (e.g., a person), an environmental source (e.g., furniture, a plant, walls, etc.), or a combination thereof. The microphone array may include a single microphone, more than one microphone, or a plurality of microphones. Each microphone of the microphone array may receive a reflected acoustic signaled in response to the provided acoustic signal reflecting off the subject.

In some examples, receive beamforming techniques may be implemented to aid in contactless motion tracking. More specifically, receive beamforming techniques may be implemented to generate a beamformed signal and determine the location of the subject (e.g., localization). In some examples, the receive beamforming techniques may be based at least on performing a search over multiple angles to locate a selected angle based on a signal strength of the motion data. In some examples, the selected angle may be selected to maximize the signal strength of the motion data. In other examples, the selected angle may by selected to meet or exceed a quality threshold.

In other examples, the receive beamforming techniques may be based at least on a ternary-search performed by changing a search range as well as a beam width to compute a direction of the subject (e.g., the motion source, environmental source, etc.). In even further examples, the receive beamforming techniques may be based at least on a computation that starts at lower frequencies to reduce an effect of direction for the subject, and utilizes higher frequencies to increase beam resolution and selected a direction of the subject. In some examples, the computation may be a divide and conquer technique.

In some embodiments, the speaker may be physically coupled to the microphone array. In further embodiments, the speaker may not be physically coupled to the microphone array but collocated with the microphone array. In even further examples, the speaker may neither be physically coupled to the microphone array nor collocated with the microphone array. In some examples, synchronization may occur between the speaker and the microphone array, where the synchronization is based at least on regenerating the provided acoustic signal using a known seed, performing cross-correlation between the received reflected acoustic signal and the regenerated provided acoustic signal resulting in a cross-correlation output, and identifying a peak of the cross-correlation output, where the peak is indicative of and/or corresponds to a direct path from the speaker to the microphone array.

Examples of computing devices described herein may extract motion data of the subject based at least in part on the received reflected acoustic signal. In some examples, motion data may be extracted by transforming the received reflected acoustic signal into a structured signal based at least in part of shifting a phase of each frequency component of the received reflected acoustic signal. In some examples, the structured signal is a frequency-modulated continuous wave (FMCW) chirp. The structured signal may be demodulated based at least on multiplying the structured signal by a conjugate signal. The demodulated structured signal may be decoded based at least on performing a fast Fourier transformation (FFT). Motion data may be extracted using phase information corresponding to the FFT frequency bin of the decoded demodulated structured signal.

In other examples, computing devices described herein may extract motion data without transforming the received reflected acoustic signal into a structured signal. Here, the computing device may determine a value of a FFT frequency bin corresponding to an estimated round-trip distance of the received reflected acoustic signal. At least a respiratory motion signal may be determined using the value of the FFT frequency bin. A continuous phase signal (e.g., phase information to extract motion data) may be extracted by applying sub-band merging and phase shift compensation to the respiratory signal.

In even further examples, computing devices described herein may extract motion data using a machine-learning and/or pattern recognition techniques. In some cases the machine-learning model is a convolutional neural network (CNN), a deep convolutional neural network (DCNN), a recurrent neural network (RNN), or any other type of neural network, or combination thereof. In some cases, the motion data extracted from the subject may be or include a respiratory motion signal, a coarse movement motion signal, a respiration rate, health condition information, and the like. In other cases, the motion data extracted from the subject may be or include any other data indicative of health and/or sleep conditions and/or anomalies. In some examples, the motion data extracted from the subject may be used to identify at least one health condition, such as, for example, congenital ENT anomalies, gastrointestinal-related health conditions, as well as neurological- and musculoskeletal-related conditions, etc.

Advantageously, systems and methods described herein utilize contactless motion tracking for monitoring motion data. Examples of such contactless motion tracking systems and methods not only facilitate more compressive motion tracking of a subject that may both improve sleep quality and identify important breathing or other anomalies, examples of such systems may be safer and less invasive than what is currently available. In addition to being contactless, safer, and capable of tracking a more comprehensive set of motion data, examples of systems and methods described herein may provide a single, commercially available device (e.g., speaker, smart speaker, smart phone, tablet, etc.) to integrate the described contactless motion tracking functionality, resulting in a reduced number of monitoring devices, the elimination of physician-assisted sleep studies, a reduction in cost, and the ability to comprehensively, contactless, and safely monitor motion in your own home. While various advantages of example systems and methods have been described, it is to be understood that not all examples described herein may have all, or even any, of the described advantages.

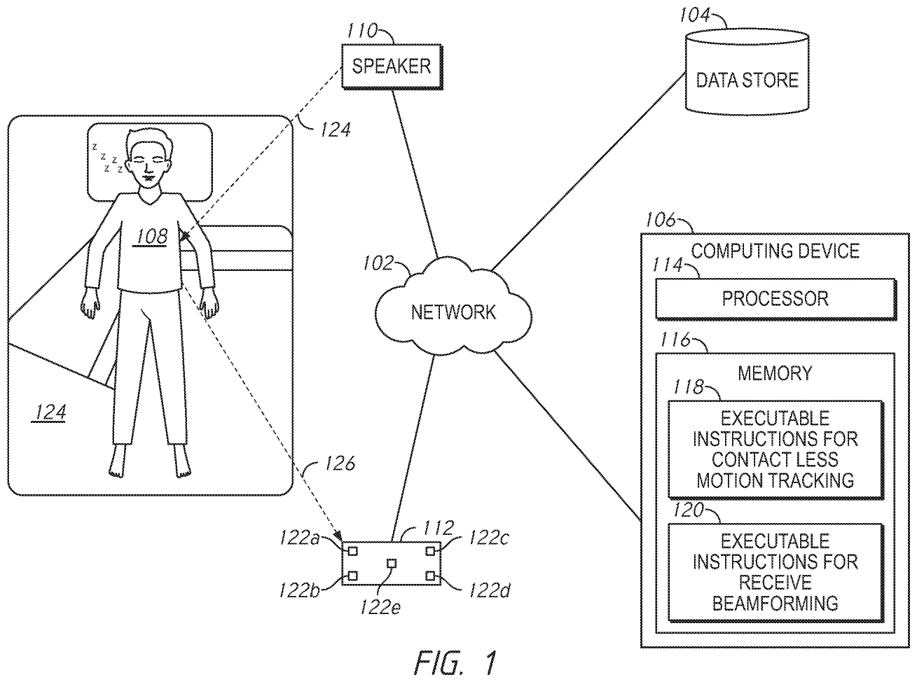

FIG. 1 is a schematic illustration of a system 100 for contactless motion tracking (e.g., a contactless motion tracking system), in accordance with examples described herein. It should be understood that this and other arrangements and elements (e.g., machines, interfaces, function, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more components may be carried out by firmware, hardware, and/or software. For instance, and as described herein, various functions may be carried out by a processor executing instructions stored in memory.

Among other components not shown, system 100 of FIG. 1 includes data store 104, computing device 106, speaker 110, and microphone array 112. Computing device 106 includes processor 114, and memory 116. Memory 116 includes executable instructions for contactless motion tracking 118 and executable instructions for receive beamforming 120. Microphone array 112 includes microphones 122a-122e. It should be understood that system 100 shown if FIG. 1 is an example of one suitable architecture for implementing certain aspects of the present disclosure. Additional, fewer, and/or different components may be used in other examples. It should be noted that implementations of the present disclosure are equally applicable to other types of devices such as mobile computing devices and devices accepting gesture, touch, and/or voice input. Any and all such variations, and any combination thereof, are contemplated to be within the scope of implementations of the present disclosure. Further, although illustrated as separate components of computing device 106, any number of components can be used to perform the functionality described herein. Although illustrated as being a part of computing device 106, the components can be distributed via any number of devices. For example, processor 114 can be provided via one device, sever, or cluster of servers, while memory 116 may be provided via another device, server, or cluster of servers.

As shown in FIG. 1, computing device 106, speaker 110, and microphone array 112 may communicate with each other via network 102, which may include, without limitation, one or more local area networks (LANs) and/or wide area networks (WANs). Such networking environments are commonplace in offices, enterprise-wide computer networks, laboratories, homes, intranets, and the Internet. Accordingly, network 102 is not further described herein. It should be understood that any number of computing devices and/or visual stimulus devices may be employed within system 100 within the scope of implementations of the present disclosure. Each may comprise a single device or multiple devices cooperating in a distributed environment. For instance, computing device 106 could be provided by multiple server devices collectively providing the functionality of computing device 106 as described herein. Additionally, other components not shown may also be included within the network environment.

Computing device 106, speaker 110, and microphone array 112 have access network 102) to at least one data store or repository, such as data store 104, which includes any data related to generating, providing, and/or receiving acoustic signals, various receive beamforming techniques described herein, various motion data extraction techniques described herein, as well as the any metadata associated therewith. In implementations of the present disclosure, the data store is configured to be searchable for one or more of the data related to generating, providing, and/or receiving acoustic signals, the various receive beamforming techniques, and/or the motion data extraction techniques described herein.

Such information stored in data store 104 may be accessible to any component of system 100. The content and volume of such information are not intended to limit the scope of aspects of the present technology in any way. Further, data store 104 may be a single, independent component (as shown) or a plurality of storage devices, for instance, a database cluster, portions of which may reside in association with computing device 106, speaker 110, microphone array 112, another external computing device (not shown), and/or any combination thereof. Additionally, data store 104 may include a plurality of unrelated data repositories or sources within the scope of embodiments of the present technology. Data store 104 may be local to computing device 106, speaker 110, or microphone array 112. Data store 104 may be updated at any time, including an increase and/or decrease in the amount and/or types of data related to generating, providing, and/or receiving acoustic signals, various receive beamforming techniques described herein, various motion data extraction techniques described herein (as well as all accompanying metadata).

Examples of speaker 110 described herein may generally implement providing acoustic signals, such as signal 126 of FIG. 1. In some examples, the acoustic signals may be audible signals, inaudible signals, or a combination thereof. In some examples, the acoustic signals may be pseudorandom signals. Examples of pseudorandom signals described herein include random signals. Pseudorandom signals generally refer to signals that exhibit statistical randomness (e.g., may not be easily predicted). Some examples of pseudorandom signals described herein include signals generated using information which may be secret, hidden, and/or difficult to acquire. Examples of pseudorandom signals include white noise signals, Gaussian white noise signals, brown noise signals, pink noise signals, wide-band signals, narrow-band signals, or combinations thereof. In some examples, speaker 110 may generate a pseudorandom white noise signal based on pseudo-random sequences and a known seed, such that a generated pseudorandom white noise signal has a flat frequency response. In some examples, speaker 110 may use an impulse signal encoded by shifting the phases of each of its frequency components by a random real sequence uniformly distributed in [0, 2π] to generate a pseudorandom white noise signal. In other examples, speaker 110 may generate a pseudorandom signal by generating a pseudorandom number sequence and normalize the amplitude of each frequency component. As should be appreciated, while only two methods are discussed for generating a pseudorandom white noise signal, this is in no way limiting, and other methods for generating pseudorandom signals (e.g., pseudorandom white noise signals) are contemplated to be within the scope of this disclosure.

In some examples, the signal generated by speaker 110 may follow Gaussian white noise for the following reasons. First, an impulse signal is flat in the frequency domain, and randomly changing the phase of each of its frequency components does not affect this. Further, the pseudorandom phase, denoted by ϕ_{f}, is independent and uniformly distributed in [0, 2π]. From the central limit theorem, suppose a sampling rate is r, and each time-domain sample,

$$\frac{1}{\sqrt{r / 2}} {\sum }_{f = 1}^{r / 2} exp ( - j ( 2 π f t + {∅}_{f} ) ,$$

follows a normal distribution with a zero mean and constant variance when r is large enough, making it Gaussian white noise. As should be appreciated, other white noise generating techniques that provide these features may also be used. Moreover, in other examples, other signal sources may additionally and/or alternatively be used.

In some examples, speaker 110 may generate a signal as a stream of blocks, each of which having a constant duration. A long duration may provide for an increase in signal to noise ratio (SNR) of the received reflected acoustic signal using correlation. In one example, a duration of T=0.2 s and a sampling rate of 48000 Hz is used; so, the frequency range is 1 Hz to fₘₐₓ=24000 Hz. As should be appreciated, other time durations and sampling rates may also be used, and this example is in no way limiting.

In some embodiments, speaker 110 may be used to provide acoustic signals to a subject, such as, for example, a motion source, an environmental source, and the like. As used herein, a motion source may include a person (e.g., an adult, an infant, a child, etc.), such as motion source 108 of FIG. 1. As used herein, an environmental source may include furniture, walls, plants, and the like, such as bed 124 of FIG. As should be appreciated, speaker 110 may be implemented using any number of audio devices, including but not limited to, a speaker, a smart speaker, white noise machine, and the like. In some examples, speaker 110 may be integrated into a computing device, such as a smartphone, tablet, other handheld device, computer, and the like.

Examples of microphone array 112 described herein may generally implement receiving reflected acoustic signals, such as reflected signal 128 of FIG. 1. Microphone array 112 may include microphones 122a-122e. While five microphones are shown in FIG. 1, generally any number of microphones may be included in a microphone array described herein. Moreover, microphones 122a-122e are depicted in FIG. 1 as arranged at each of the four corners and in the center of microphone array 112, however, other arrangements of microphones may be used in other examples. Microphones 122a-122e may receive reflected acoustic signals, such as reflected signal 128, responsive to a provided acoustic signal, such as acoustic signal 126 provided by speaker 110, reflecting off of a subject (e.g., a motion source, such as motion source 108, and/or an environmental source, such as bed 124). Microphone array may be communicatively coupled to a computing device, such as computing device 106, that is capable of contactless motion tracking in accordance with examples described herein. Microphone array 112 may also be communicatively coupled to a speaker, such as speaker 110, that is capable of providing acoustic signals, as described herein.

Examples described herein may include computing devices, such as computing device 106 of FIG. 1. Computing device 106 may in some examples be integrated with one or more speaker(s) and/or one or more microphone array(s) described herein. In some examples, computing device 106 may be implemented using one or more computers, servers, smart phones, smart devices, or tablets. Computing device 106 may facilitate contactless motion tracking, and in some examples, facilitate receive beamforming. As described herein, computing device 106 includes processor 114 and memory 116. Memory 116 includes executable instructions for contactless motion tracking 118 and executable instructions for receive beamforming 120. In some embodiments, computing device 106 may be physically coupled to speaker 110 and/or microphone array 112. In other embodiments, computing device 106 may not be physically coupled to speaker 110 and/or microphone array 112 but collocated with the speaker and/or the microphone array. In even further embodiments, computing device 106 may neither be physically coupled to speaker 110 and/or microphone array 112 nor collocated with the speaker and/or the microphone array.

Computing devices, such as computing device 106 described herein may include one or more processors, such as processor 114. Any kind and/or number of processor may be present, including one or more central processing unit(s) (CPUs), graphics processing units (CPUs), other computer processors, mobile processors, digital signal processors (DSPs), microprocessors, computer chips, and/or processing units configured to execute machine-language instructions and process data, such as executable instructions for contactless motion tracking 118 and/or executable instructions for receive beamforming 120.

Computing devices, such as computing device 106, described herein may further include memory 116. Any type or kind of memory may be present (e.g., read only memory (ROM), random access memory (RAM), solid state drive (SSD), and secure digital card (SD card). While a single box is depicted as memory 116, any number of memory devices may be present. The memory 116 may be in communication (e.g., electrically connected) to processor 114.

Memory 116 may store executable instructions for execution by the processor 114, such as executable instructions for contactless motion tracking 118 and/or executable instructions for receive beamforming 120. Processor 114, being communicatively coupled to speaker 110 and microphone array 112, and via the execution of executable instructions for contactless motion tracking 118 and/or execution of executable instructions for receive beamforming 120, may extract motion data from a subject. The extracted motion data may include respiratory motion signals, coarse movement motion signals, respiration rate, and other health condition related data. At least one health condition, sleeping disorder, etc. may be identified from the extracted motion data.

In operation, to perform contactless motion tracking, processor 114 of computing device 106, executing executable instruction for contactless motion tracking 118, may synchronize speaker 110 and microphone array 112. In some cases, to synchronize speaker 110 and microphone array 112, processor 114 may regenerate the signal provided by speaker 110 at microphone array 112 using a known seed. Processor 114 may perform a cross-correlation between the received reflected acoustic signal and the regenerated provided acoustic signal, where the result of the cross-correlation is a cross-correlation output. Based at least on the cross-correlation output, processor 114 may identify a peak of the cross-correlation output, where the peak corresponds to a direct path from speaker 110 to microphone array 112. As can be appreciated, in some examples, synchronization may only need to be performed once at the beginning of contactless motion tracking as speaker 110 and microphone array 112 may, in some cases, share the same sampling clock. However, in other cases, synchronization may need to be performed more than once, such as, for example, in the event of a lost connection between speaker 110 and microphone array 112. In even further cases, synchronization may need to be performed more than once even when connection has not be lost. In other cases, synchronization may not need to be performed at all, such as, for example, if speaker 110 and microphone array 112 are physically coupled. As should be appreciated, while cross-correlation is discussed, other forms of similarity measurements are contemplated to be within the scope of the present disclosure.

Various techniques are described herein to extract motion data of a subject, based on a received reflected acoustic signal. As one example technique, to extract motion data from a subject, processor 114 of computing device 106, executing executable instruction for contactless motion tracking 118, may transform the received reflected acoustic (e.g., pseudorandom) signal into a structured signal (e.g., structured chirp), where the transforming is based, at least in part, on shifting a phase of each frequency component of the received reflected acoustic signal. In some examples, the structured signal is a frequency-modulated continuous wave (FMCW) signal. As should be appreciated, while an FMCW chirp is described, any other structured signal is contemplated to be within the scope of this disclosure.

As should be appreciated, one advantage of transforming a received reflected acoustic signal (e.g., a white noise signal) into a structured signal (e.g., an FMCW chirp) is the transformation aids in removing and/or lessening the randomness of the reflected received acoustic signal, may allow for the reflected received acoustic signal to be more efficiently decoded to track motions (including minute motions), and aids in preventing loss of information of the reflected received acoustic signal. Moreover the transformation described herein can further preserve multipath information of received reflected acoustic signals.

For example, in the presence of multiple paths, the received reflected acoustic signal within the frequency range [f₀T, (f₀+F)T] may be written as:

$$w ( t ) = {\sum }_{p ∈ paths} {A}_{p} {\sum }_{f = {f}_{0} T}^{( {f}_{0} + F ) T} {e}^{- j ( 2 π f \frac{t - {t}_{p}}{T} + {∅}_{f} )}$$

where Aₚ and tₚ are the attenuation factor and time-of-arrival of path p. Performing a discrete Fourier transformation (DFT) on w(t), w(t) can be rewritten as:

$$W ( f ) = {\sum }_{p ∈ paths} {A}_{p} {e}^{- 2 π \frac{{t}_{p}}{T} f + {∅}_{f}} = {A}_{f}^{′} {e}^{- j {Φ}_{f}}$$

In some examples, a phase transformation disclosed herein may change the phase of each frequency as follows, \hat{Φ}_{f}=Φ_{f}−ϕ_{f}+ψ_{f}. This may, in some examples, convert the received reflected acoustic signal (e.g., white noise signal) into an FMCW chirp without losing multipath information.

Mathematically, transforming a received reflected acoustic signal (e.g., white noise signal) can be illustrated by the following:

$$\hat{w} ( t ) = {\sum }_{f = {f}_{0 T}}^{( {f}_{0} + F ) T} {\sum }_{p ∈ paths} {A}_{p} {e}^{- j ( 2 π f \frac{t - {t}_{p}}{T} + {ϕ}_{f} )} {e}^{- j ( - {ϕ}_{f} + {ψ}_{f} )} = {\sum }_{f = {f}_{0} T}^{( {f}_{0} + F ) T} {\sum }_{p ∈ paths} {A}_{p} {e}^{- j ( 2 π f \frac{t - {t}_{p}}{T} + {ψ}_{f} )} = {\sum }_{p ∈ paths} {A}_{p} {\sum }_{f = {f}_{0} T}^{( {f}_{0} + F ) T} {e}^{- j ( 2 π f \frac{t - {t}_{p}}{T} + {ψ}_{f} )} \approx \frac{1}{C} {\sum }_{p ∈ paths} {A}_{p} fmcw ( t - {t}_{p} )$$

Where the final approximation is because α_{f}≈1. As illustrated, the multipath reflections from the subject (e.g., a motion source, an environmental source, etc.) in the received reflected acoustic (e.g., white noise) signal are preserved after processor 114 transforms the received reflected acoustic signal into an FMCW chirp.

Processor 114 may demodulate the structured signal, where the demodulating is based, at least in part, on multiplying the structured signal by a conjugate signal, and where the result of the demodulating results in a demodulated signal (e.g., demodulated chirp) and at least one corresponding frequency bin. In some cases, demodulated the structured signal may enable processor 114 to separate received reflected acoustic signals that are reflected from environmental sources, from those reflected from motion sources. from other environmental sources from that of the subject,

Processor 114 may decode the demodulated signal (e.g., demodulated chirp), where the decoding is based at least in part performing a fast Fourier transformation (FFT) on the demodulated signal (e.g., demodulated chirp), resulting in at least one corresponding FFT frequency bin. Using the phase information associated with the corresponding FFT frequency bin, processor 114 may extract the motion data of the subject.

In some examples, processor 114 may transform a received reflected acoustic signal (e.g., white noise signal) into a single large FMCW chirp spans the whole frequency range (e.g., band) of the signals being provided by speaker 110. Advantageously, a large band FMCW chirp may have better spatial resolution because of the more fine-grained frequency bins after demodulation and DFT.

However, in other examples, processor 114 may, split the band into five sub-bands, which are then transformed into five concurrent FMCW chirps to be demodulated and decoded for motion extraction. Advantageously, by transforming the band into five sub-bands, and subsequently transforming the received reflected acoustic signal into five intendent FMCW chirps, overall SNR may be improved. This is because the same frequency bin of each of the five demodulated FMCW chirps corresponds to a same time-of-arrival at microphone array 112. Accordingly, the five phases of each FFT bin from each demodulated FMCW chirp may be fused thereby improving SNR. As should be appreciated, while splitting the band into five sub-bands is described, this is in no way limiting, and the band can be split into greater or fewer sub-bands, as well as remain one band.

As a further example technique, to extract motion data from a subject, processor 114 of computing device 106, executing executable instruction for contactless motion tracking 118, may determine a value of a FFT frequency bin corresponding to an estimated round-trip distance d of the received reflected acoustic signal. Using the value of the FFT frequency bin, processor 114 may determine a respiratory motion signal. Processor 114 may then extract continuous phase signal from the respiratory motion signal by applying sub-band merging and phase shift compensation.

Mathematically, processor 114 may determine the value of an FFT bin corresponding to estimated round-trip distance d as follows:

$$H ( d ) = {\sum }_{t ∈ [ 0 , T ]} {\sum }_{p ∈ paths} {A}_{p} {e}^{- j 2 π ( \frac{F}{T} {t}_{p} t + {f}_{0} {t}_{p} - \frac{F}{2 T} {t}_{p}^{2} )} * {e}^{j 2 π \frac{Fd}{Tc} t} = {\sum }_{R ∈ [ 0 , 1 ]} {\sum }_{p ∈ paths} {A}_{p} {e}^{- j 2 π ( FR ( {t}_{p} - \frac{d}{c} ) + {f}_{0} {t}_{p} - \frac{F}{2 T} {t}_{p}^{2} )}$$

It may also be assumed that due to near distance, (e.g., 1m, tp/T≈0), then

$$\frac{F \frac{{t}_{p}}{2 T}}{FR + {f}_{0}} \approx 0.$$

### Accordingly,

$$H ( d ) \approx {\sum }_{E ∈ [ 0 , 1 ]} {\sum }_{p ∈ paths} {A}_{p} {e}^{- j 2 π ( FR ( {t}_{p} - \frac{d}{c} ) + {f}_{0} {t}_{p} )}$$

Mathematically, processor 114 may determine the respiratory motion signal as follows:

$${H}^{′} ( d ) = conj ( {\sum }_{p ∈ paths} {A}_{p} {\sum }_{f = {f}_{0} T}^{( {f}_{0} + F ) T} {e}^{- j ( 2 π f \frac{t - {t}_{p}}{T} + {∅}_{f} )} * {e}^{j ( 2 π {f}_{0} \frac{d}{c} t + {∅}_{f} )} )$$

After determining the respiratory motion signal, processor 114 may apply a sub-band merging and 2π phase shift compensation as described herein, and extract the continuous phase signal.

As an even further example technique, to extract motion data from a subject, rather than extract motion data by transforming the received reflected acoustic signal into a structured signal or obtaining the phase of H(d) and/or H′(d), processor 114 of computing device 106, executing executable instruction for contactless motion tracking 118, may extract motion data using amplitude instead. In operation, processor 114 may feed amplitude information, phase information, or a combination thereof, corresponding to the received reflected acoustic signal into a neural network, where the neural network is configured to compress the amplitude information, phase information, or the combination thereof, from a two-dimension (2D) space into a one-dimensional (1D) space. Based at least on the compressed amplitude information, phase information, or a combination thereof, processor 114 may extract the motion data of the subject. In some examples, the neural network is a convolutional neural network (CNN). In other examples, the neural network is a deep convolutional neural network (DCNN). In even further examples, the neural network is a recurrent neural network (RNN), or any other type of neural network, or combination thereof.

As should be appreciated, while only three motion data extraction techniques are described herein, additional and/or alternative motion data extraction techniques are contemplated without departing from the scope of the present disclosure.

In some examples, receive beamforming may be implemented to assist in contactless motion tracking, and in particular, localize the subject. In operation, to localize the subject, processor 114 of computing device 106, executing executable instruction for receive beamforming 120, beamform the received reflected acoustic signal to generate a beam formed signal. Processor 114 may determine a location of the subject based at least in part on the beamforming.

In some examples, receive beamforming may be implemented to assist in contactless motion tracking after localization. In operation, processor 114 may perform beamforming based on at least a determined distance between a subject and the speaker, a determined beamforming signal, a determined angle of the subject relative to the speaker, or a combination thereof. In some examples, determining the angle of the subject relative to the speaker is based at least on performing a search over multiple angles to locate a selected angle based on a signal strength of the motion data. In other examples, determining the angle of the subject relative to the speaker is based at least on a ternary-search performed by changing a search range as well as a beam width to compute a direction of the subject. In even further examples, determining the angle of the subject relative to the speaker is based at least on a computation that starts at lower frequencies to reduce an effect of direction for the subject, and utilizes higher frequencies to increase beam resolution and select a direction of the subject

In some examples, extracted motion data comprises respiration motion (e.g., breathing motion), coarse movement motion (e.g., leg movement, arm movement, etc.), respiration rate (e.g., breathing rate), sound (e.g., crying, etc.) and the like. Based at least on the extracted motion data, processor 114 may identify at least one health condition, breathing condition, neuromuscular condition, sleep disorder, sleep abnormality, sleep anomaly, and the like, that may be used to determine a corrective recommendation.

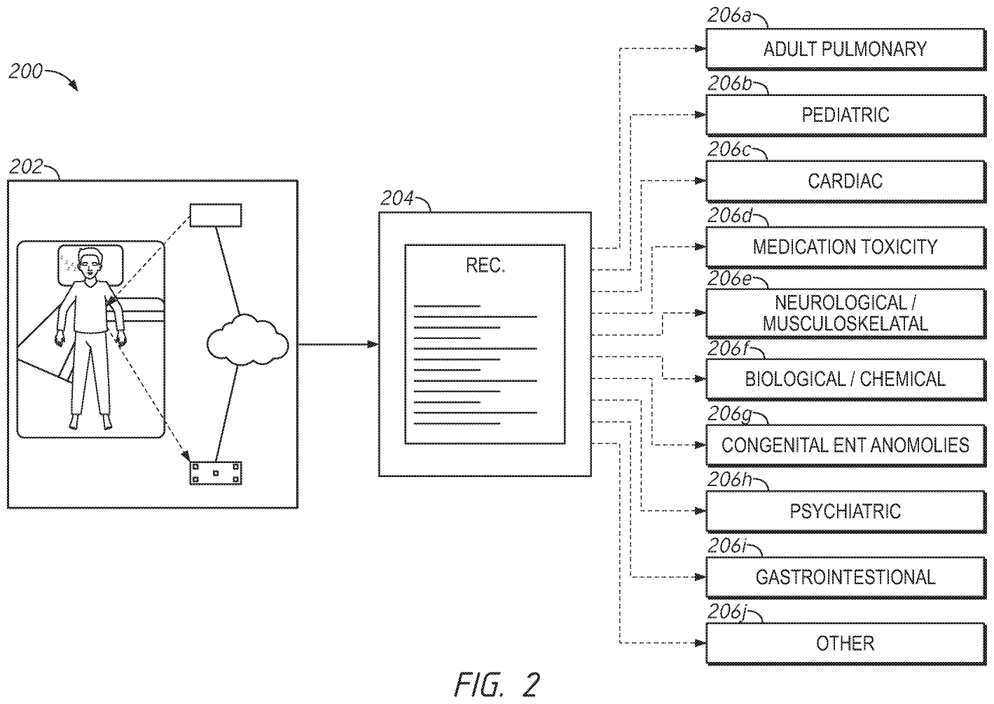

Turing now to FIG. 2, FIG. 2 illustrates a schematic illustration of using contactless motion tracking results for identification of health conditions and medical correction, in accordance with examples described herein. FIG. 2 includes contactless motion tracking block 202, recommendation block 204, and health condition identification type blocks 206a-206j.

In examples described herein, contactless motion tracking system 100 may be used to identify sleep abnormalities and/or other health conditions. In operation, and at contactless motion tracking block 202, motion may be tracked by providing, by a speaker, an acoustic signal. In some examples, the acoustic signal is a pseudorandom signal. A microphone array may receive a reflected acoustic signal based on the provided acoustic signal reflecting off a subject, such as, for example, a motion source (e.g., a person), an environmental source (e.g., furniture, a plant, walls, etc.), or a combination thereof. In some examples, receive beamforming techniques may be used to aid in the localization of the subject and the detection of the reflected acoustic signal. Motion data (e.g., respiratory motion, coarse movement motion, respiration rate, and the like) may be extracted from the subject using the received reflected acoustic signal based at least on various extraction techniques described herein.

Based at least on the extracted motion data, and as can be seen at recommendation block 204, the contactless motion tracking system may make a recommendation about corrective treatment for at least one identified health condition or sleep anomaly. Examples of possible identified health conditions or sleep anomalies can be seen at health condition identification type blocks 206a-206j. For example, health conditions that contactless motion system 100 may identify and/or provide corrective treatment recommendations may include, but is not limited to, adult pulmonary health condition 206a, pediatric health condition 206b, cardiac health condition 206c, medication toxicity health condition 206d, neurological/musculoskeletal health condition 206e, biological/chemical health condition 206f, congenital ENT anomaly health condition 206g, psychiatric health condition 206h, gastrointestinal health condition 206i, as well as other health conditions 206j.

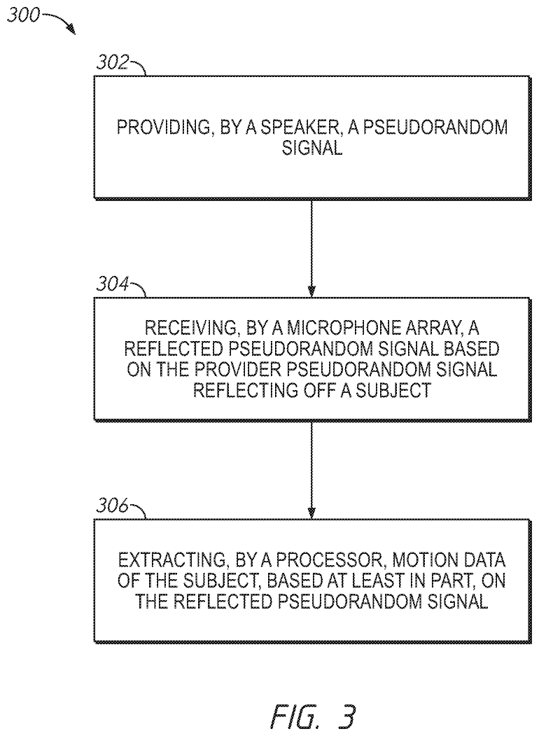

FIG. 3 is a flowchart of a method 300 arranged in accordance with examples described herein. The method 300 may be implemented, for example, using system 100 of FIG. 1.

The method 300 includes providing, by a speaker, a pseudorandom signal at block 302, receiving, by a microphone array, a reflected pseudorandom signal based on the provided pseudorandom signal reflecting off a subject at block 304, and extracting, by a processor, motion data of the subject, based at least in part, on the reflected pseudorandom signal at block 306.

Block 302 recites providing, by a speaker, a pseudorandom (e.g., acoustic) signal. In one embodiment the pseudorandom signal may be an audible signal, an inaudible signal, or a combination thereof. In a further embodiment, the pseudorandom signal may be a white noise signal, a Gaussian white noise signal, a brown noise signal, a pink noise signal, a wide-band signal, a narrow-band signal, or any other pseudorandom signal.

Block 304 recites receiving, by a microphone array, a reflected pseudorandom signal based on the provided pseudorandom signal reflecting off a subject. In some embodiments, the subject may be a motion source (e.g., a person), an environmental source (e.g., furniture, a plant, walls, etc.), or a combination thereof. The microphone array may include a single microphone, or a plurality of microphones. Each microphone of the microphone array may receive a reflected acoustic signaled in response to the provided acoustic signal reflecting off the subject.

Block 306 recites extracting, by a processor, motion data of the subject, based at least in part, on the reflected pseudorandom signal. Generally, motion data may be extracted by reversing (e.g., undoing) some or all of the randomness in the reflected pseudorandom signal. In some embodiments, motion data may be extracted by transforming the received reflected pseudorandom signal into a structured signal based at leak in part of shifting a phase of each frequency component of the received reflected pseudorandom signal. In some examples, the structured signal is a frequency-modulated continuous wave (FMCW) chirp. The structured signal may be demodulated based at leak on multiplying the structured signal by a conjugate signal. The demodulated structured signal may be decoded based at least on performing a fast Fourier transformation (FFT). Motion data may be extracted using phase information corresponding to the FFT frequency bin of the decoded demodulated structured signal.

In other embodiments, computing devices described herein may extract motion data without transforming the received reflected pseudorandom signal into a structured signal. Here, the computing device may determine a value of a FFT frequency bin corresponding to an estimated round-trip distance of the received reflected acoustic signal. At least a respiratory motion signal may be determined using the value of the FFT frequency bin. A continuous phase signal (e.g., phase information to extract motion data) may be extracted by applying sub-band merging and phase shift compensation to the respiratory signal. In even further examples, computing devices described herein may extract motion data using a machine-learning and/or pattern recognition techniques.

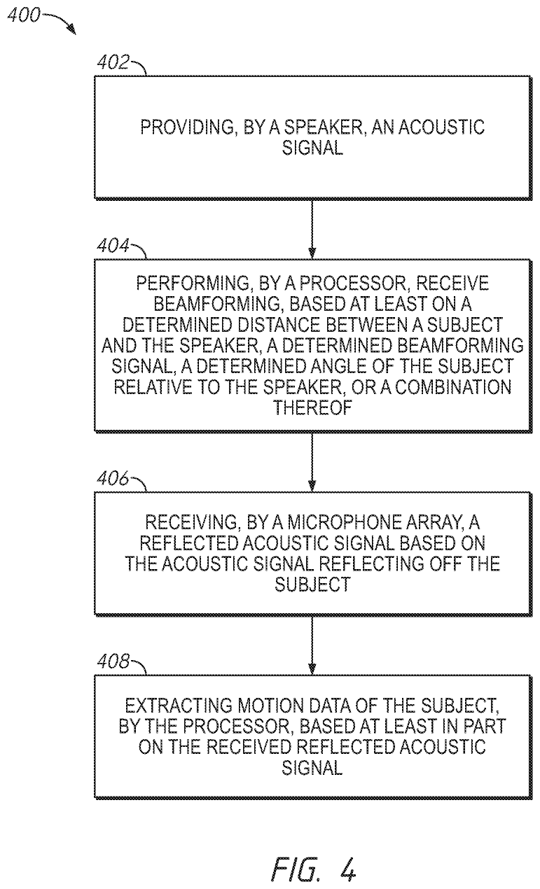

FIG. 4 is a flowchart of a method 400 arranged in accordance with examples described herein. The method 400 may be implemented, for example, using the system 100 of FIG. 1.

The method 400 includes providing, by a speaker, an acoustic signal at block 402, performing, by a processor, receive beamforming, based at least on a determined distance between a subject and the speaker, a determined beamforming signal, a determined angle of the subject relative to the speaker, or a combination thereof at block 404, receiving, by a microphone array, a reflected acoustic signal based on the acoustic signal reflecting off the subject at block 406, and extracting motion data of the subject, by the processor, based at least in part, on the received reflected acoustic signal at block 408.

Block 402 recites providing, by a speaker, an acoustic signal. In one embodiment the acoustic signal may be an audible signal, an inaudible signal, or a combination thereof. In a further embodiment, the acoustic signal may be a pseudorandom signal. In even further examples, the acoustic signal may be a white noise signal, a Gaussian white noise signal, a brown noise signal, a pink noise signal, a wide-band signal, a narrow-band signal, or any other pseudorandom signal.

Block 404 recites performing, by a processor, receive beamforming, based at least on a determined distance between a subject and the speaker, a determined beamforming signal, a determined angle of the subject relative to the speaker, or a combination thereof. In some embodiments, the receive beamforming techniques may be based at least on performing a search over multiple angles to locate a selected angle based on a signal strength of the motion data. In some embodiments, the selected angle may be selected to maximize the signal strength of the motion data. In other embodiments, the selected angle may by selected to meet or exceed a quality threshold.

In other embodiments, the receive beamforming techniques may be based at least on a ternary-search performed by changing a search range as well as a beam width to compute a direction of the subject (e.g., the motion source, environmental source. etc.). In even further embodiments, the receive beamforming techniques may be based at least on a computation that starts at lower frequencies to reduce an effect of direction for the subject, and utilizes higher frequencies to increase beam resolution and selected a direction of the subject. In some embodiments, the computation may be a divide and conquer technique.

Block 406 recites receiving, by a microphone array, a reflected acoustic signal based on the acoustic signal reflecting off the subject. In some embodiments, the subject may be a motion source (e.g., a person), an environmental source (e.g., furniture, a plant, walls, etc.), or a combination thereof. The microphone array may include a single microphone, or a plurality of microphones. Each microphone of the microphone array may receive a reflected acoustic signaled in response to the provided acoustic signal reflecting off the subject.

Block 408 recites extracting motion data of the subject, by the processor, based at leak in part, on the received reflected acoustic signal. In some embodiments, motion data may be extracted by transforming the received reflected pseudorandom signal into a structured signal based at least in part of shifting a phase of each frequency component of the received reflected pseudorandom signal. In some examples, the structured signal is a frequency-modulated continuous wave (FMCW) chirp. The structured signal may be demodulated based at least on multiplying the structured signal by a conjugate. The demodulated structured signal may be decoded based at least on performing a fast Fourier transformation (FFT). Motion data may be extracted using phase information corresponding to the FFT frequency bin of the decoded demodulated structured signal.

In some embodiments, computing devices described herein may extract motion data without transforming the received reflected pseudorandom signal into a structured signal. Here, the computing device may determine a value of a FFT frequency bin corresponding to an estimated round-trip distance of the received reflected acoustic signal. At least a respiratory motion signal may be determined using the value of the FFT frequency bin. A continuous phase signal (e.g., phase information to extract motion data) may be extracted by applying sub-band merging and phase shift compensation to the respiratory signal. In even further examples, computing devices described herein may extract motion data using a machine-learning and/or pattern recognition techniques.

Once motion data is obtained using systems and/or techniques described herein, any of a variety of actions may be taken using the motion data. The motion data may be displayed, for example on a monitor or wearable device. In some examples, the motion data may be used to generate an alarm if the motion data meets a predetermined criteria for the motion data. The motion data may be transmitted to other device(s) (e.g., a device of a medical practitioner). The motion data may be used to diagnose a particular medical condition.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

### IMPLEMENTED EXAMPLES

### Evaluation

In an evaluation of an example implementation of the contactless motion tracking system 100 described herein, a smart speaker prototype, built with a MiniDSP UMA-8-SP USB microphone array, and which was equipped with 7 Knowles SPH1668LM4H microphones, was used. The smart speaker prototype was connected to an external speaker PUI AS07104PO-R), and a plastic case that holds the microphone array and speaker together was 3D-printed. The microphone array was connected to a Surface Pro laptop. Dynamically generated pseudo-random white noise was played and the 7-channel recordings were recorded, using XT-Audio library. The acoustic signals were captured at a sampling rate of 48 kHz and 24 bits per sample.

Next, the effectiveness and accuracy of an example implementation of the contactless motion tracking system 100 described herein was evaluated. Extensive experiments were conducted with a tetherless newborn simulator. The simulator, designed to train physicians on neonatal resuscitation, mimics the physiology of newborn infants. The effect of different parameters, including recording position, orientation and distances, at-ear sound pressure level, interference from other people, respiration strength and rate was systematically evaluated. Five infants at a Neonatal Intensive Care Unit (NICU) were then recruited and a clinical study was conducted to verify the validity of the contactless motion tracking system 100 described herein on monitoring respiration, motion and crying.

### Neonatal Simulator Experiments

Because of the experimental difficulty of placing a wired ground truth monitor on a healthy sleeping infant, an infant simulator (SimNewB®, Laerdal, Stavanger, Norway), co-created by the American Academy of Pediatrics, that mimics the physiology of newborn infants was used first. SimNewB is a tetherless newborn simulator designed to help train physicians on neonatal resuscitation and is focused on the physiological response in the first 10 minutes of life. It comes with an anatomically realistic airway and supports various breathing features including bilateral and unilateral chest rise and fall, normal and abnormal breath sounds, spontaneous breathing, anterior lung sounds, unilateral breath sounds and oxygen saturation. These life-like simulator mannequins, which retail >$25,000, are used to train medical personnel on identifying vital sign abnormalities in infants, including respiratory anomalies. SimNewB is operated and controlled by SimPad PLUS, which is a wireless tablet. Various parameters of the simulator are controllable, including a) respiration rate and intensity; b) limb motion; and c) sound generation. The controllable parameters were used to evaluate different aspects of BreathJunior's performance.

Specifically, experiments were performed in the simulator lab in a medical school where an infant simulator was put in a 26 inch×32 inch bassinette by one of the walls shown in FIG. 8. The smart speaker prototype (described herein) was placed on a stand that can adjust the orientation, and put the stand on a table which can adjust its position around the crib. Its height was set to 10 cm above the simulator so that the rails of the bassinette will not obstruct the path between the prototype and the simulator.

### Effect of Distance, Orientation, and Position

With respect to smart speaker position, the effect of the smart speaker position with respect to the infant on breathing rate accuracy was measured first. To do this, the smart speaker hardware was placed in four different positions around the bassinette: left, right, front and rear. This effectively evaluates the effect of placing the smart speaker at different sides of a crib. The smart speaker was placed at different distances from the chest of the infant, from 30 cm to 60 cm. At each of the distances, the infant simulator was set to breathe at a breathing rate of 40 breaths per minute, which is right in the middle of the expected breathing rate for infants. As the default, the sound pressure was set to be 56 dB at the infant's ear. The smart speaker transmits the white noise signal and the acoustic signals were recorded for one minute, which was then use to compute the breathing rate. This experiment was repeated ten times.

Key trends were, first, the average computed respiratory rate across the distances up to 60 cm is around 40 breaths per minute, which is the configured breathing rate of the infant simulator (shown by the dotted line). Second, the position of the smart speaker does not significantly affect the breathing error rate. The only exception is when the smart speaker is placed at the rear, where we have slightly higher variance in the measured breathing rate. This is because there is more obstruction from the abdomen and legs. Finally, as expected, the variance in the measured breathing rate increases with distance. Specifically, the mean absolute error is around 3 breaths per minute when the smart speaker is at a distance of 60 cm, compared to 0.4 breaths per minute at a distance of 40 cm. This is because the reflections from the infant's breathing motion attenuate with distance.

With respect to smart speaker orientation, experiments were next run with three different smart speaker orientations. This allows an evaluation of the effectiveness of beamforming as a function of the smart speaker angle. The breathing rate of the simulator was set to 40 BPM and vary the distance of the smart speaker from the infant's chest. The at-ear sound pressure was set to 56 dB. The results showed that there is no significant difference in the respiratory rate variance across the three orientations. This is because the microphone array (e.g., microphone array 112 of FIG. 1) is designed to be omni-directional to detect sound across all angles.

### Effect of Volume, Respiration Rate, and Intensity

Next, the effect of sound volume, respiration rate and intensity on breathing rate accuracy was evaluated.

With respect to smart speaker sound volume, the higher the sound volume from the smart speaker, the better the reflections from the infant breathing motion. However, in some applications, the target is to keep the white noise volume to be under 60 dB at-ear to be conservatively safe. Here, the effect of different at-ear white noise volumes was evaluated. Specifically, the white-noise volume was changed to be between 50-59 dB(A). As before the distance between the smart speaker and the infant simulator was changed between 30-70 cm and measure the breathing rate using the white noise reflections at each of these volume levels. The smart speaker is placed at the left and 0° with respect to the infant. As before, the experiment was repeated ten times to compute the mean and variance in the estimated breathing rate while the simulator is set to a breathing rate of 40 breaths per minute.

The results show that when the at-ear sound volume is around 56 dB(A), low variance in the breathing rate estimation up to distances of 50 cm was achieved. When the white noise volume at the infant was increased by 3 dB to 59 dB(A), the breathing rate can be estimated with low variance from a distance of up to 70 cm. This is expected since the reflections from the breathing motion are stronger when the white noise volume is higher.

With respect to respiration rate and intensity, the accuracy of the system with varying respiration rates as well as the intensity of each breath was evaluated. For a typical infant less than one year old, the respiration ate is less than 60 breaths per minute. So, the accuracy was evaluated by varying the breathing rate of the infant simulator between 20-60 breaths per minute. To verify the robustness, the intensity of each breath on the simulator to two different settings: normal and weak, was also changed. The weak intensity is triggered by a simulated respiratory distress syndrome (RDS), an ailment that can be experienced by infants and particularly those born prematurely. The distance of the infant simulator from the smart speaker was set to 40 cm and the speaker was placed at the left and at 0°.

The results of these experiments with the smart speaker-computed breathing rate as a function of the simulator breathing setting. Also noted are the results for the two intensity settings. The plots show that there was a higher variance in the computed breathing rate as the breathing rate was increased. This is because, as the breathing rate increases, more changes within the received signal are seen, which requires higher sampling rates to get the same error resolution. In implementations, the block of each white noise signal was set to 0.2 s. Thus, as the breathing rate increases, less blocks per each breath are seen, which effectively reduces the number of samples per breath, which in turn introduces more errors. As expected, more variance is seen in weak breath situations associated with respiratory distress syndrome. This is because lower intensity results in smaller phase change, resulting in a lower SNR.

### Effect of Clothes and Interference

Finally, the effect of blankets and other interfering motion in the environment was evaluated.

With respect to clothes, a typical infant one-piece sleep sack made of cotton which is provided with the simulator to help trainees learn the correct method for putting on this garment that helps swaddle the baby was used. The experiments were repeated with and without the sleep sack. Experiments were run by placing the smart speaker to the left of the infant simulator and at an angle of 0°, while setting the simulator to breathe at a rate of 40 breaths per minute. The distance was changed between the simulator and the smart speaker and compute the breathing rate. The results show that the presence of sleep sack does not significantly affect the breathing rate accuracy. The system disclosed herein was further evaluated with human infants who are swaddled in blankets in described herein and show that the system can track their breathing motion.

With respect to interference, the above experiments are all done when an adult is sitting about three meters away from the crib. To further assess if the interference from other people would affect the accuracy, the same experiments additionally run with an adult sitting at consecutively closer distances. The results show there is not much difference except when the distance between the adult and the smart speaker is 1 meter, while the distance between the simulator and the smart speaker is 60 cm, since the small distance difference leads to spectrum leakage in the FFT of the FMCW demodulation. However, the system disclosed herein could still extract a breathing rate at this distance.

### Effect of Receive Beamforming

Here, the benefits of using receive beamforming were quantitatively evaluated. As described herein, experiments were run by placing the smart speaker to the left of the infant simulator and at an angle of 0°, while setting the simulator to breathe at a rate of 40 breaths per minute. At-ear sound pressure was kept at 59 dB and change the distance of the smart speaker and the infant simulator and collect the data on the smart speaker. The breathing signals were then extracted using a single microphone on the smart speaker to decode the signal in the absence of our receive beamforming algorithm. The receive beamforming was then run. The results show that receive beamforming improves the range by approximately 1.5-2×, which is approximately a 5 dB SNR gain.

### Apnea Motion, and Sound Detection

Here the ability of the system disclosed herein to identify apnea events, body motion as well as audible sound is evaluated.

With respect to apnea detection, an apnea event is defined as a 15-second respiratory pause. While it is difficult to run experiments with human infants that also have apnea events, they can be simulated on the infant simulator described herein. Specifically, a 15 second central apnea event is simulated by remotely pausing the respiration of the infant simulator and resuming it after 15 seconds. The thresholding method described herein was used to detect the presence of an apnea event during the 15 second. The 15-second duration was used before the apnea event where the infant simulator breathes normally to evaluate the false positive rate (FP). The smart speaker was placed 50 cm left of the simulator at an angle of zero degree. The simulator is set to breathe at a rate of 40 breaths per minute. This experiment was repeated 20 times to generate the receiver operating characteristic (ROC) curve by different values of the threshold by computing the sensitivity and specificity of the algorithm in identifying apnea events. As expected, the sensitivity and specificity improve at higher volume.

With respect to motion detection, the ability of the system disclosed herein to detection body movements such as hand and leg motion was evaluated. The infant simulator can be remotely controlled to move its arms and legs. Specifically, for each movement, the arm or leg rotates around the shoulder joint away from the body for an angle of approximately 30°, than rotates back to its original position. Each movement takes approximately two seconds. Each of these movements are performed 20 times and record the true positive events. Like before, 20 2-second clips of normal breathing motion under the same condition were used. The distance between the infant simulator and the smart speaker was set to 50 cm and the simulator was set to breath at 40 breaths per minute.

Results show the ROC curves for each of the three movements: arm motion, leg motion and arm+leg motion. The AUC for the three movements was 0.9925, 0.995 and 1 respectively. The plots show that the system's accuracy for motion detection is high. For instance, the operating point for arm motion had an overall sensitivity and specificity of 95% (95% CI: 75.13% to 99.87%) and 100% (95% CI: 83.16% to 100.00%), respectively. This is expected because these movements reflect more power than the minute breathing motion and hence can be readily identified.

Finally, the ability of the system disclosed herein to detect infant audible sounds was evaluated. The infant simulator has an internal speaker that plays realistic recorded sounds of infant crying, coughing and screaming, which are frequent sounds from infants. The volume is to set to be similar to an infant sound. As before, 20 2-second clops of each sound type and use 20 2-second clips where the simulator was breathing but was silent were recorded. The infant simulator was set to breathe at 40 BPM and the distance from the smart speaker was 60 cm.

### Neonatal Simulator Experiments

The American Academy of Pediatrics strongly recommends against any wired systems in an infant's sleep environment, making ground truth collection of respiratory signals on healthy infants at home unsafe and potentially ethically challenging. To overcome this challenge, clinical studies are conducted at the Neonatal Intensive Care Unit (NICU) of a major medical center. The vast majority of infants in this NICU are born prematurely (i.e., before 38 weeks gestation). This environment was chosen because the infants are all connected to wired, hospital-grade respiratory monitors providing ground truth while they sleep in their bassinets. Each infant is treated in individual bassinets in a separate room, where their parents and nurses are also sitting around 1.5 meters away from the bassinet, most of the time. Five infants were recruited, with consent from their parents, over the course of a month. This study was approved by our organization's Institutional Review Board and followed all the prescribed criteria.

### Clinical Study Setup

Since infants at this age sleep intermittently between feedings, the recording sessions ranged from 20 minutes to 50 minutes. All infants, because they were in the NICU, were connected to hospital grade respiratory monitoring equipment (Phillips LTD). The smart speaker prototype is placed outside the crib to ensure safety, and the distance between the prototype and the monitored infant is kept between 40-50 cm. The at-ear sound pressure is 59 dB(A). 7 total session are performed over a total duration of 280 minutes. Of these, the nurses or parents were interacting or feeding the infant for 62 minutes. The techniques are performed over the remaining 218 minutes.

### Respiratory Rate Accuracy

Respiratory rate measurements from the Phillips hospital system was accessible with minute-to-minute granularity. The clocks between the logging computer in the hospital and a laptop were synchronized to align the start of each minute. Note that the precision of the groundtruth respiratory rate is 1 BPM. Since the target population is infants above the age of 1 month, infants who have a weight more than 3.5 kg which is the average weight of a newborn infant were focused on.

### Motion and Crying Detection Accuracy

Finally, the capabilities of the system described herein for motion and sound detection are compared with the ground truth. The threshold values from the simulator experiments which gave us the best sensitivity and specificity for this purpose was used. The duration was manually noted, on a minute resolution, when the infant is crying and moving; this was used as the ground truth for these experiments. The results show that there is a good correlation with the ground truth.

### Clinical Use Cases

As described throughout, contactless motion tracking system 100 may identify health conditions, etc. using extracted motion data. Below is a non-limiting list of various clinical uses cases for system 100 identifying health condition.

| | Sonar (audible/inaudible) | Sound (audible) |
|---|---|---|
| Adult Pulmonary | | |
| COPD | breathing frequency, | cough, expectoration |
| | cough, heart rate, | |
| | nocturnal awakenings | |
| Central Apnea | breathing frequency, apnea | |
| Sleep apnea syndrome | breathing frequency, | snoring, apnea, post- |
| | apnea ,hypopnea, | apnea gasp |
| Asthma | breathing frequency, | cough, wheeze |
| | cough heart rate | |
| CF | breathing frequency, cough | |
| Infectious disease | breathing frequency, cough, | cough |
| (flu, URI, cold) | nocturnal awakenings, rigors | |
| Pediatric | | |
| Apnea, apnea of | breathing frequency, | |
| prematurity | apnea, heart rate | |
| Respiratory infection | breathing frequency, | cough, cough characteristics |
| | cough, heart rate | (barking cough, stridor) |
| Neonatal sepsis | breathing frequency, | cough, cry |
| | heart rate, cough | |
| Pertussis | cough | whooping cough; |
| | | cough paroxysm |
| Croup | cough | barking cough |
| Asthma | breathing frequency, | cough, wheeze |
| | cough, heart rate | |
| Acute chest syndrome | breathing frequency | |
| Cystic fibrosis exacerbation | breathing frequency, cough | cough, wheeze, expectoration |
| Cardiac | | |
| Congestive heart | respiratory rate, pulse, | cough |
| failure (CHF) | increased JVP, cough | |
| Bradycardia, tachycardia | heart beat | n/a |
| Cardiac arrest (ventricular | heart beat; apnea | agonal breathing |
| fibrillation, aystole, v-tach) | | |
| Other arythmeas (afib, SVT, | heart beat, respiratory rate | |
| a-flutter, AVNRT + more) | (from shortness of breath) | |
| Neurological/ | | |
| musculoskeletal | | |
| Stroke | | slurred speech |
| Seizure | seizure motion (tonic/clonic) | seizure sound |
| Parkinson's | tremor | speech change, shuffling gait |
| ALS, MSA | forwards dropping of the head | |
| | (disproportionate antecollis) | |
| Fall | fall motion | fall sound |
| Pain | heart rate, respiratory rate | |
| Medication toxicity | | |
| Opioids, benzodiazapenes, | breathing frequency, apnea | agonal breathing |
| gabapentin | | (if leads to arrest) |
| Post-operative home setting | breathing frequency, apnea | agonal breathing |
| (respiratory depressant | | (if leads to arrest) |
| medications) | | |
| Ace-inhibitor | | cough |
| Biologic or | | |
| chemical weapons | | |
| (Anthrax, tularemia, | tachypnea, respiratory | cough, gasp |
| phosgene, nitrogen mustard, | failure, cough | |
| nerve agents, ricin) | | |
| Congenital ENT anomalies | | |
| Nasal, craniofacial | stridor | stridor, wheezing |
| tongue anomalies, , | | |
| laryngomalacia | | |
| laryngeal (webs, | | |
| cysts, clefts) | | |
| Subglottic hemangioma, | stridor | stridor |
| stenosis, tracheal stenosis, | | |
| vascular rings, | | |
| tracheomalacia, | | |
| bronchogenic cyst | | |
| Psychiatric | | |
| Anxiety, panic attack | respiratory rate | |
| Depression | sleep disturbance | voice change |
| PTSD | sleep disturbance, | |
| | respiratory rate | |
| Gastrointestinal | | |
| Reflux | cough | voice change, cough |
| Laryngopharyngeal reflux | cough | voice change, cough |
| Swallowing dysfunction/ | cough | cough |
| silent aspiration | | |
| Other | | |
| Malaria | rigors, respiratory rate, | |
| | nocturnal awakenings | |

1. A system comprising:
a speaker configured to provide a pseudorandom signal;
a microphone array configured to receive a reflected pseudorandom signal based on the provided pseudorandom signal, wherein the received reflected pseudorandom signal is responsive to the provided pseudorandom signal reflecting off a subject; and
a processor configured to extract motion data of the subject, based at least in part, on the received reflected pseudorandom signal.

2. The system of claim 1, wherein the pseudorandom signal comprises an acoustic signal, and wherein the pseudorandom signal comprises at least one of a white noise signal, a Gaussian white noise signal, a brown noise signal, a pink noise signal, a wide-band signal, a narrow-band signal, or combinations thereof.

3. (canceled)

4. The system of claim 1, wherein the motion data comprises at least one of a respiratory motion signal, a coarse movement motion signal, a respiration rate, a health condition, or a combination thereof.

5. The system of claim 1, wherein the speaker is further configured to generate the pseudorandom signal, based, at least in part, on a phase-shift encoded impulse signal.

6. The system of claim 1, wherein the processor is further configured to synchronize the speaker and the microphone array.

7. The system of claim 6, wherein the processor is further configured to synchronize the speaker and the microphone array based at least in part on:
regenerating the provided pseudorandom signal using a known seed;
performing cross-correlation between the received reflected pseudorandom signal and the regenerated provided pseudorandom signal, wherein the performing results in a cross-correlation output; and
identifying a peak of the cross-correlation output, wherein the peak corresponds to a direct path from the speaker to the microphone array.

8. The system of claim 1, wherein the processor is further configured to localize the subject based at least in part on determining a distance from the speaker to the subject.

9. The system of claim 8, wherein the processor is further configured to localize the subject based, at least in part, on beamforming the received reflected pseudorandom signal, received at the microphone array, to generate a beamformed signal, and determining a location of the subject, based at least in part, on the beamforming.

10. The system of claim 1, wherein the processor is further configured to extract the motion data based at least on:
transforming the received reflected pseudorandom signal into a structured signal, wherein the transforming is based, at least in part, on shifting a phase of each frequency component of the received reflected pseudorandom signal, shifting a frequency of each component of the received reflected pseudorandom signal, or a combination thereof;
demodulating the structured signal, wherein the demodulating is based, at least in part, on multiplying the structured signal by a conjugate signal, wherein the demodulating results in a demodulated signal and at least one corresponding frequency bin;
decoding the demodulated signal, wherein the decoding is based, at least in part, on performing a fast Fourier transformation (FFT) on the demodulated signal, resulting in at least one corresponding FFT frequency bin; and
extracting, using phase information associated with the corresponding FFT frequency bin, the motion data of the subject.

11. The system of claim 10, wherein the structured signal is a frequency-modulated continuous wave (FMCW) signal.

12. The system of claim 1, wherein the processor is further configured to extract the motion data based at least on:
determining a value of a FFT frequency bin corresponding to an estimated round-trip distance of the received reflected pseudorandom signal;
using the value of the FFT frequency bin, determine a respiratory motion signal;
and applying sub-band merging and phase shift compensation to extract a continuous phase signal.

13. The system of claim 1, wherein the processor is further configured to extract the motion data based at least on:
feeding amplitude information, phase information, or a combination thereof, corresponding to the received reflected pseudorandom signal into a neural network, wherein the neural network is configured to compress the amplitude information and the phase information from a two-dimension (2D) space into a one-dimensional (1D) space; and
based at least on the compressed amplitude information, phase information, or a combination thereof, extracting the motion data of the subject.

14. The system of claim 13, wherein the neural network comprises at least one of a convolutional neural network, a deep convolutional neural network, a recurrent neural network, or combinations thereof.

15. The system of claim 1, wherein the processor is further configured to identify at least one health condition based at least on extracting the motion data of the subject.

16. A method comprising:
providing, by a speaker, a pseudorandom signal;
receiving, by a microphone array, a reflected pseudorandom signal based on the provided pseudorandom signal reflecting off a subject; and
extracting, by a processor, motion data of the subject, based at least in part, on the reflected pseudorandom signal.

17. The method of claim 16, wherein the pseudorandom signal comprises an acoustic signal, and wherein the pseudorandom signal comprises at least one of a white noise signal, a Gaussian white noise signal, a brown noise signal, a pink noise signal, a wide-band signal, a narrow-band signal, and wherein the pseudorandom signal comprises at least one of an audible signal, an inaudible signal, or combinations thereof.

18. The method of claim 16, wherein the motion data comprises at least one of a respiratory motion signal, a coarse movement motion signal, a respiration rate, a health condition, or a combination thereof.

19. The method of claim 16, further comprising:
synchronizing, by the processor, the speaker and the microphone array, based at least on, regenerating the provided pseudorandom signal using a known seed,
performing cross-correlation between the received reflected pseudorandom signal and the regenerated provided pseudorandom signal, wherein the performing results in a cross-correlation output, and
identifying a peak of the cross-correlation output, wherein the peak corresponds to a direct path from the speaker to the microphone array.

20. (canceled)

21. The method of claim 16, wherein extracting motion data comprises:
transforming, by the processor, the received reflected pseudorandom signal into a structured signal, wherein the transforming is based, at least in part, on shifting a phase of each frequency component of the received reflected pseudorandom signal, shifting a frequency of each component of the received reflected pseudorandom signal, or a combination thereof;
demodulating, by the processor, the structured signal, wherein the demodulating is based, at least in part, on multiplying the structured signal by a conjugate signal, wherein the demodulating results in a demodulated signal and at least one corresponding frequency bin;
decoding, by the processor, the demodulated signal, wherein the decoding is based, at least in part, on performing a fast Fourier transformation (FFT) on the demodulated signal, resulting in at least one corresponding FFT frequency bin; and
extracting, by the processor, using phase information associated with the corresponding FFT frequency bin, the motion data of the subject.

22. The method of claim 16, wherein extracting the motion data comprises:
determining, by the processor, a value of a FFT frequency bin corresponding to an estimated round-trip distance of the received reflected pseudorandom signal;
using, by the processor, the value of the FFT frequency bin, determine a respiratory motion signal; and
applying, by the processor, sub-band merging and phase shift compensation to extract a continuous phase signal.

23.-30. (canceled)
